(19) United States
(12) Patent Application Publication
(10) Pub. No.: US 2021/0087582 A1
(43) Pub. Date: Mar. 25, 2021

(54) METHOD AND COMPOSITION FOR ENDOGENOUS PRODUCTION OF CONSTITUTIVELY ACTIVATED RECEPTORS, AND RECEPTORS WITH BROADER BINDING RANGES OR HIGHER AFFINITY THAN NATIVE RECEPTORS

(71) Applicant: Delors Pharma Inc., Calgary (CA)

(72) Inventor: Bradley G. THOMPSON, Calgary (CA)

(73) Assignee: Delors Pharma Inc.

(21) Appl. No.: 16/580,996

(22) Filed: Sep. 24, 2019

(51) Int. Cl. C12N 15/86; C12N 7/00; A61K 35/76; A61K 48/00; A61K 38/17

(52) U.S. Cl. CPC C12N 15/86; C12N 7/00; C12N 2750/14143; A61K 48/0008; A61K 38/177; A61K 35/76

(57) ABSTRACT

The present disclosure relates to one or more agents, therapies, treatments, and methods of use of the agents and/or therapies and/or treatments for upregulating the production of one or more receptors proteins that are constitutively activated, or have broader binding ranges or higher affinity than native receptors. Embodiments of the present disclosure can be used as a therapy or a treatment for a subject that has a condition whereby the subject's receptor-ligand system is, or is likely to become, dysregulated and wherein the upregulation of these proteins may be of therapeutic benefit.

### TECHNICAL FIELD

The present disclosure generally relates to molecules that are receptor proteins within a subject. In particular, the present disclosure relates to compositions, and/or the production, of one or more receptor proteins that are constitutively activated, or have broader binding ranges or higher affinity than native receptors.

### BACKGROUND

Biological receptors comprise one or more proteins that can cause a change in cellular activity in response to stimuli. The stimuli can be any number of environmental stimuli (internal or external) such as, but not limited to: chemical stimuli, thermal stimuli, light stimuli, pressure stimuli and combinations thereof. Chemical stimuli often comes in the form of a molecule, referred to as a ligand, which has a specific shape to bind with the receptor.

Receptors can be positioned upon a cell's phospholipid membrane in order to receive some ligands. Receptors can also be positioned between cells and within cells.

When a receptor binds a ligand, the receptor can have a biological effect directly in the cell or other cells. The biological effect can alter the effected-cells' production and/or functionality of genes, proteins and/or signal molecules for example by increasing, decreasing or maintaining the transcription, translation, and post-translational functions of the effected cells.

Mu (μ) opioid receptors (MOR) can bind a variety of opioid molecules as ligands. MORs are positioned upon the surface of pre-synaptic neurons, post-synaptic cells and within the spinal cord. Binding of an opioid ligand and a MOR has many physiological effects on the subject, such as feelings of euphoria, sedation and analgesia.

The analgesic effect of opioids makes them effective and widely used for treating patients suffering from pain but they also have well known side-effects, such as increased tolerance and increased dependence.

### SUMMARY

Some embodiments of the present disclosure relate to a method of making an agent/target cell complex, the method comprising a step of administering a therapeutically effective amount of the agent to a subject, wherein the agent/target cell complex increases the subject's production of one or more receptor proteins that are constitutively activated, or have broader binding ranges or higher affinity than native receptors.

Some embodiments of the present disclosure relate to a method of making an agent/target cell complex, the method comprising a step of administering a sufficient amount of an agent to a target cell whereby the agent/target cell complex is formed, wherein the agent/target cell complex increases the production of one or more receptor proteins that are constitutively activated, or have broader binding ranges or higher affinity than native receptors by said target cell.

Some embodiments of the present disclosure relate to a pharmaceutical agent that comprises an agent, a pharmaceutically acceptable carrier and/or an excipient. The agent may upregulate production one or more receptor proteins that are constitutively activated, or have broader binding ranges or higher affinity than native receptors.

Some embodiments of the present disclosure relate to a method of treating a condition. The method comprises a step of administering to a subject a therapeutically effective amount of an agent that upregulates the production one or more receptor proteins that are constitutively activated, or have broader binding ranges or higher affinity than native receptors.

Some embodiments of the present disclosure relate to a use of an agent for treating a condition, wherein the agent upregulates the one or more receptor proteins that are constitutively activated, or have broader binding ranges or higher affinity than native receptors in a subject that receives the agent.

Some embodiments of the present disclosure relate to a method for upregulating the production one or more receptor proteins that are constitutively activated, or have broader binding ranges or higher affinity than native receptors, the method comprising a step of making an agent/target cell complex.

Embodiments of the present disclosure relate to at least one approach for inducing endogenous production of one or more receptor proteins that are constitutively activated, or have broader binding ranges or higher affinity than native receptors.

A first approach utilizes gene vectors containing nucleotide sequences and/or genes for one or more receptor proteins that are constitutively activated, or have broader binding ranges or higher affinity than native receptors, which can be administered to subjects to increase the production of one or more receptor proteins that are constitutively activated, or have broader binding ranges or higher affinity than native receptors.

Without being bound by any particular theory, embodiments of the present disclosure may be useful for treating conditions wherein the ligand-receptor function of a patient is, or is likely to become, dysregulated. In some embodiments of the present disclosure relate to upregulating the production and/or functionality of one or more receptor proteins that are constitutively activated. In some embodiments of the present disclosure relate to upregulating the production and/or functionality of one or more receptor proteins that have broader binding ranges compared to native receptor proteins. In some embodiments of the present disclosure relate to upregulating the production and/or functionality of one or more receptor proteins that have a higher affinity compared to native receptor proteins. The embodiments of the present disclosure may be used as a therapy or a treatment for a subject that has a condition whereby any of the subject's ligand-receptor function is, or is likely to become, dysregulated. A non-limiting example of such a dysregulated ligand-receptor function is when the subject has developed one or both of tolerance and dependence upon an opioid.

### DETAILED DESCRIPTION

### Definitions

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of biomolecule.

As used herein, the term “affinity” refers to the intra-molecular, attractive forces—such as Van der Waals forces, a potential for forming ionic bonds and a potential for forming hydrogen bonds—between a receptor and a given ligand. A higher affinity may cause a ligand to be bound with the receptor for a longer amount of time, as compared to when there is a lower affinity.

As used herein, the term “agent” refers to a substance that, when administered to a subject, causes one or more chemical reactions and/or one or more physical reactions and/or one or more physiological reactions and/or one or more immunological reactions in the subject.

As used herein, the term “ameliorate” refers to improve and/or to make better and/or to make more satisfactory.

As used herein, the term “biomolecule” refers to a carbohydrate, a protein, an amino acid sequence, a nucleic acid, a lipid, a primary metabolite, a secondary metabolite that is found within a subject. A biomolecule may be endogenous or exogenous to a subject.

As used herein, the expression “broader binding range” refers to the ability of a receptor according to the embodiments of the present disclosure to induce a change in the effected cells' production and/or functionality of genes, proteins and/or other signalling molecules in the presence of a lower amount of a ligand, as compared to a native receptor.

As used herein, the term “cell” refers to a single cell as well as a plurality of cells or a population of the same cell type or different cell types. Administering an agent to a cell includes in vivo, in vitro and ex vivo administrations and/or combinations thereof.

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, and/or any post-translational modifications of one or more proteins.

As used herein, the expression “constitutively activate” refers to the ability of a receptor to cause a change in the cellular activity of one or more cells when the receptor is substantially free of any bound ligand.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “effector molecule” refers to a molecule within a subject that can directly or indirectly regulate the metabolic activity of a target cell by increasing or decreasing the production of DNA, RNA and/or amino-acid sequences and/or by increasing or decreasing any post-translational modifications of one or more proteins.

As used herein, the term “endogenous” refers to the production and/or modification of a molecule that originates within a subject.

As used herein, the term “excipient” refers to any substance, not itself an agent, which may be used as a component within a pharmaceutical composition or a medicament for administration of a therapeutically effective amount of the agent to a subject. Additionally or alternatively, an excipient may, either alone or in combination with further chemical components, improve the handling and/or storage properties, and/or permit or facilitate formation of a dose unit, of the agent. Excipients include, but are not limited to, one or more of: a binder, a disintegrant, a diluent, a buffer, a taste enhancer, a solvent, a thickening agent, a gelling agent, a penetration enhancer, a solubilizing agent, a wetting agent, an antioxidant, a preservative, a surface active agent, a lubricant, an emollient, a substance that is added to mask or counteract a disagreeable odor, fragrances or taste, a substance added to improve appearance or texture of the composition and/or a substance that is used to form the pharmaceutical compositions or medicaments. Any such excipients can be used in any dosage forms according to the present disclosure. The foregoing classes of excipients are not meant to be exhaustive but are provided merely to be illustrative of what a person of skill in the art would know and would also recognize that additional types and combinations of excipients may be used to achieve delivery of a therapeutically effective amount of the agent to a subject through one or more routes of administration.

As used herein, the term “exogenous” refers to a molecule that is within a subject but that did not originate within the subject.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises the agent and that can promote recovery from a disease, disorder or symptom thereof and/or that can prevent a disease, disorder or symptom thereof and/or that can inhibit the progression of a disease, disorder, or symptom thereof.

As used herein, the term “patient” refers to a subject that is afflicted with a disease or disorder. The term “patient” includes human and veterinary subjects.

As used herein, the term “pharmaceutical composition” means any composition comprising, but not necessarily limited to, an agent to be administered a subject in need of therapy or treatment for a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render said formulation isotonic. Examples of suitable pharmaceutically acceptable carriers include, but are not limited to: saline solutions, glycerol solutions, ethanol, N-(1(2, 3-dioleyloxy)propyl)-N, N, Ntrimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention of” and “preventing” refer to avoiding the onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of an amino acid sequence, and/or the production of one or more regulatory molecules that can influence the production and/or functionality of an effector molecule or an effector cell. For clarity, “production” is also be used herein to refer to the functionality of a regulatory molecule, unless the context reasonably indicates otherwise.

As used herein, the terms “promote”, “promotion”, and “promoting” refer to an increase in an activity, response, condition, disease process, or other biological parameter. This can include, but is not limited to, the initiation of the activity, response, condition, or disease process. This may also include, for example, a 10% increase in the activity, response, condition, or disease as compared to the native or control level. Thus, the increase in an activity, response, condition, disease, or other biological parameter can be 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence and/or progression of the disease or disorder within the subject.

As used herein, the terms “signal molecule”, “signalling molecule” and “regulatory molecule” can be used interchangeably and refer to a molecule that can directly or indirectly affect the production and/or functionality of an effector molecule or effector cell. Signal molecules can be enzymes or other types of biomolecules that can act as a direct ligand on a target cell or they may influence the levels or functionality of a downstream ligand or a receptor for a ligand.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cells and/or cell types that are deleteriously affected, either directly or indirectly, by a dysregulated ligand-receptor function and/or a disease process.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In one embodiment of the present disclosure, the pharmaceutical compositions disclosed herein comprise an agent as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%. about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The present disclosure relates to one or more agents, therapies, treatments, and methods of use of the agents and/or therapies and/or treatments for upregulating the production of one or more receptor proteins that are constitutively activated, or have broader binding ranges or higher affinity than native receptors. Some embodiments of the present disclosure relate to methods for making a complex between at least one particle of an agent and at least one target cell of a subject. The complex upregulates the subject's production of one or more receptor proteins that are constitutively activated, or have broader binding ranges or higher affinity than native receptors and/or the complex upregulates the subject's production and/or functionality of one or more or the production of one or more receptor proteins that are constitutively activated, or have broader binding ranges or higher affinity than native receptors. Embodiments of the present disclosure can be used as a therapy or a treatment for a subject that has a condition whereby the subject's receptor-ligand function is, or is likely to become, dysregulated.

In some embodiments of the present disclosure, the agent can be administered to the subject by an intravenous route, an intramuscular route, an intraperitoneal route, an intrathecal route, an intravesical route, a topical route, an intranasal route, a transmucosal route, a pulmonary route, and combinations thereof.

In some embodiments of the present disclosure, the agent can be administered to the subject by pipetting a dose of the agent into an in vitro cell culture, perfusing or immersing an ex vivo cell or tissue preparation with a solution that comprises the agent, mixing a biological fluid sample with a solution or substrate that comprises the agent, or combinations thereof.

Some embodiments of the present disclosure relate to an agent that can be administered to a subject with a condition. When a therapeutically effective amount of the agent is administered to the subject, the subject may change production and/or functionality of one or more receptor proteins that are constitutively activated, or have broader binding ranges or higher affinity than native receptors. For example, the subject may increase or decrease production and/or functionality of one or signaling molecules and/or one or more effector molecules by changing the production of one or more sequences of DNA, one or more sequences of RNA and/or one or more proteins and/or one or more regulatory molecules that regulate the levels and/or functionality of the subject's signaling molecules and/or effector molecules.

In some embodiments of the present disclosure, the subject may respond to receiving the therapeutic amount of the agent by changing production and/or functionality of one or more intermediary molecules by changing production of one or more DNA sequences, one or more RNA sequences, and/or one or more proteins that regulate the levels and/or functionality of the one or more intermediary molecules. The one or more intermediary molecules regulate the subject's levels and/or functionality of the one or more signaling molecules and/or the one or more effector molecules.

In some embodiments of the present disclosure, administering a therapeutic amount of the agent to a subject upregulates the production of one or more receptor proteins that are constitutively activated, or have broader binding ranges or higher affinity than native receptors and the agent upregulates the production, functionality or both of one or more regulatory molecules, of one or more receptor proteins that are constitutively activated, or have broader binding ranges or higher affinity than native receptors. The agent can upregulate production of the one or more receptor proteins that are constitutively activated, or have broader binding ranges or higher affinity than native receptors and regulatory molecules of one or more receptor proteins that are constitutively activated, or have broader binding ranges or higher affinity than native receptors by increasing one or more of: synthesis of one or more nucleotides, nucleosides, sequences or genes that are related to increased amounts, of one or more receptors proteins that are constitutively activated, or have broader binding ranges or higher affinity than native receptors; transcription of RNA that is related to increased amounts or functionality of one or more receptors proteins that are constitutively activated, or have broader binding ranges or higher affinity than native receptors; or translation of one or more amino acids or amino acid sequences that are related to increased amounts of one or more receptors proteins that are constitutively activated, or have broader binding ranges or higher affinity than native receptors. Examples of one or more receptor proteins that are constitutively activated, or have broader binding ranges or higher affinity than native receptors that the agent can upregulate the production of include, but are not limited to: opiate receptor proteins, including the mu, delta, kappa, zeta, and nociceptin receptor proteins; dopamine receptor proteins; serotonin receptor proteins; or combinations thereof.

In some embodiments of the present disclosure, administering a therapeutic amount of the agent to a subject upregulates the production, functionality or both of one or more regulatory molecules that regulate the production or functionality of one or one or more receptor proteins that are constitutively activated or have broader binding ranges or higher affinity than native receptors and/or one or more regulatory molecules of one or more receptor proteins that are constitutively activated, or have broader binding ranges or higher affinity than native receptors. The one or more regulatory molecules can be a sequence of DNA, RNA or amino acids that causes an increase in the production of one or more receptor proteins that are constitutively activated, or have broader binding ranges or higher affinity than native receptors and regulatory molecules of one or more receptor proteins that are constitutively activated, or have broader binding ranges or higher affinity than native receptors after administration of the agent. The agent can upregulate the production or functionality of the one or more regulatory molecules by increasing one or more of: synthesis of one or more nucleotides, nucleosides, sequences or genes that are related to stimulating or otherwise causing increased amounts or functionality of the one or more regulatory molecules; transcription of RNA that is related to increased amounts or functionality of the one or more regulatory molecules; or translation of one or more amino acids or amino acid sequences that are related to stimulating or otherwise causing increased amounts or functionality of the one or more regulatory molecules. Examples of such regulatory molecules are a sequence of DNA or a sequence of RNA that causes increased amounts of one or more receptor proteins that are constitutively activated, or have broader binding ranges or higher affinity than native receptors.

In some embodiments of the present disclosure, the agent is a vector used for gene therapy. The gene therapy is useful for increasing the production of one or more receptor proteins that are constitutively activated, or have broader binding ranges or higher affinity than native receptors and regulatory molecules of receptors. For example, the vector can contain a gene that causes increased expression of the opiate receptor proteins including the mu, delta, kappa, zeta, and nociceptin receptor proteins, dopamine receptor proteins, or serotonin receptor proteins, and combinations thereof.

In some embodiments of the present disclosure, the vector used for gene therapy is a virus vector or a recombinant virus vector that can be within one or more of the following genera: flavivirus, influenza virus, enterovirus, rotavirus, rubellavirus, rubivirus, morbillivirus, orthopoxvirus, varicellovirus, dependoparvovirus, alphabaculovirus, betabaculovirus, deltabaculovirus, gam mabaculovirus, mastadenovirus, simplexvirus, varicellovirus, cytomegalovirus, or combinations thereof.

The embodiments of the present disclosure also relate to administering a therapeutically effective amount of the agent. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure, the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). In some embodiments, the therapeutically effective amount of the agent is between about 10 and about 1×10¹⁶ TCP/kg.

Some embodiments of the present disclosure relate to a method for making a complex within a subject. The method comprises a step of administering a therapeutically effective amount of the agent to the subject. The complex comprises at least one particle of agent and one or more target cells. When the complex is formed, it affects a change in metabolism of the one or more target cells, which results in the subject upregulating the production of one or more receptors proteins that are constitutively activated, or have broader binding ranges or higher affinity than native receptors and/or one or regulatory molecules of one or more receptor proteins that are constitutively activated, or have broader binding ranges or higher affinity than native receptors. Examples of a target cell include, but are not limited to: an adrenal gland cell; a B cell; a bile duct cell; a chondrocyte; a cochlear cell; a corneal cell; an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; a uveal cell; or combinations thereof.

Some embodiments of the present disclosure relate to a therapy that can be administered to a subject with the condition. The therapy comprises a step of administering to the subject a therapeutically effective amount of an agent that will upregulate production or activity of one or more regulatory molecules and/or one or more receptor proteins that are constitutively activated, or have broader binding ranges or higher affinity than native receptors and/or one or more regulatory molecules of one or more receptors. When the therapy is administered to a patient, the therapy will promote the in vivo production and/or functionality of one or more receptor proteins that are constitutively activated, or have broader binding ranges or higher affinity than native receptors and/or one or more regulatory molecules of one or more receptors. The increased production and/or functionality of one or more receptor proteins that are constitutively activated, or have broader binding ranges or higher affinity than native receptors, may reduce deleterious effects of the condition upon the patient.

Some embodiments of the present disclosure relate to a method of treating a condition wherein the method comprises a step of administering to the subject a therapeutically effective amount of an agent that will upregulate production or activity of one or more receptor proteins that are constitutively activated, or have broader binding ranges or higher affinity than native receptors and/or one or more regulatory molecules of one or more receptors.

Some embodiments of the present disclosure relate to one receptor proteins that are constitutively activated of at least a portion of the following amino acid sequence:

```
SEQUENCE ID 001:
  10 20 30 40
MDSSAAPTNA SNCTDALAYS SCSPAPSPGS WVNLSHLDGN
  50 60 70 80
LSDPCGPNRT DLGGRDSLCP PTGSPSMITA ITIMALYSIV
                90 100 110 120
CVVGLFGNFL VMYVIVRYTK MKTATNIYIF NLALADALAT
              130 140 150 160
STLPFQSVNY LMGTWPFGTI LCKIVISIDY YNMFTSIFTL
              170 180 190 200
CTMSVDRYIA VCHPVKALDF RTPRNAKIIN VCNWILSSAI
              210 220 230 240
GLPVMFMATT KYRQGSIDCT LTFSHPTWYW ENLLKICVFI
              250 260 270 280
FAFIMPVLII TVCYGLMILR LKSVRMLSGS KEKDRNLRKI
              290 300 310 320
TRMVLVVVAV FIVCWTPIHI YVIIKALVTI PETTFQTVSW
              320 340 350 360
HFCIALGYTN SCLNPVLYAF LDENFKRCFR EFCIPTSSNI
              370 380 390 400
EQQNSTRIRQ NTRDHPSTAN TVDRTNHQLE NLEAETAPLP
```

### Example 1

In one example, the agent is a recombinant virus vector such as an AAV6.2FF gene vector that comprises a gene insert for the gene responsible for upregulating the production of a constitutively activated mu receptor protein in humans.

In this example, the gene insert for the constitutively activated mu opioid receptor protein produces a biological compound from the following amino acid sequence for the constitutively activated mu opioid receptor protein (SEQ ID NO. 1):

```
  10 20 30 40
MDSSAAPTNA SNCTDALAYS SCSPAPSPGS WVNLSHLDGN
  50 60 70 80
LSDPCGPNRT DLGGRDSLCP PTGSPSMITA ITIMALYSIV
                90 100 110 120
CVVGLFGNFL VMYVIVRYTK MKTATNIYIF NLALADALAT
              130 140 150 160
STLPFQSVNY LMGTWPFGTI LCKIVISIDY YNMFTSIFTL
              170 180 190 200
CTMSVDRYIA VCHPVKALDF RTPRNAKIIN VCNWILSSAI
              210 220 230 240
GLPVMFMATT KYRQGSIDCT LTFSHPTWYW ENLLKICVFI
              250 260 270 280
FAFIMPVLII TVCYGLMILR LKSVRMLSGS KEKDRNLRKI
              290 300 310 320
TRMVLVVVAV FIVCWTPIHI YVIIKALVTI PETTFQTVSW
              330 340 350 360
HFCIALGYTN SCLNPVLYAF LDENFKRCFR EFCIPTSSNI
              370 380 390 400
EQQNSTRIRQ NTRDHPSTAN TVDRTNHQLE NLEAETAPLP
```

The invention claimed is:

1. A recombinant virus vector (RVV) comprising a virus with a gene insert coding for the production, of one or more receptor proteins that are constitutively activated, or have broader binding ranges or higher affinity than native receptors.

2. The RVV of claim one comprising a virus with a gene insert coding for the production of a constitutively activated mu receptor (SEQ ID No. 1).

3. A recombinant virus vector (RVV) comprising a virus with a gene insert coding for the production of a peptide comprising ten or more amino acids in a sequence of SEQ ID NO. 1.

4. The RVV of claim 1, claim 2, or claim 3 wherein the RVV is of a genus that is one or more of a flavivirus, an influenza virus, an enterovirus, a rotavirus, a rubellavirus, a rubivirus, a morbillivirus, an orthopoxvirus, a varicellovirus, a dependoparvovirus, an alphabaculovirus, a betabaculovirus, a deltabaculovirus, a gammabaculovirus, a mastadenovirus, a rubulavirus, a simplexvirus, a varicellovirus, a vesiculovirus, a lyssavirus, a cytomegalovirus and combinations thereof.

5. A method of making an agent/target cell complex, the method comprising a step of administering a recombinant virus vector (RVV) to a target cell for forming the agent/target cell complex, wherein the agent/target cell complex causes the target cell to increase production of a peptide sequence of one or more receptor proteins that are constitutively activated, or have broader binding ranges or higher affinity than native receptors.

6. The method of claim 5, wherein the target cell is one or more of an adrenal gland cell; a B cell; a bile duct cell; a chondrocyte; a cochlear cell; a corneal cell; a dendritic cell, an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a stem cell, a T cell; a testicular tissue cell; a thyroid gland cell; an uveal cell; and/or combinations thereof.

7. A pharmaceutical agent comprising:
(a) an agent that upregulates production of one or more peptide sequences of one or more receptor proteins that are constitutively activated, or have broader binding ranges or higher affinity than native receptors;
(b) a pharmaceutically acceptable carrier; and/or
(c) an excipient.

8. The pharmaceutical agent of claim 7, wherein the pharmaceutical agent is in a solid form or a fluid form.

9. A method of treating a condition, the method comprising a step of administering to a subject a therapeutically effective amount of an agent for upregulating the subject's production of one or more proteins and/or peptides of one or more receptor proteins that are constitutively activated, or have broader binding ranges or higher affinity than native receptors.

10. The method according to claim 9, where the protein and/or peptide is of SEQ ID No. 1.

11. The method according to claim 9, where the one or more receptor proteins that are constitutively activated, or have broader binding ranges or higher affinity than native receptors, are opiate receptor proteins including the mu, delta, kappa, zeta, and nociceptin receptor proteins, dopamine receptor proteins, serotonin receptor proteins, or combinations thereof.

12. The method according to claim 7, wherein the step of administering the agent occurs by an intravenous route, an intramuscular route, an intraocular route, an intraperitoneal route, an intrathecal route, an intravesical route, a topical route, an intranasal route, a transmucosal route, a pulmonary route, or combinations thereof.

13. The method according to claim 7, wherein the therapeutically effective amount is between about 10 to about 1×10¹⁶ TCID₅₀/kg of the patient's body weight.

14. The method according to claim 7, wherein the therapeutically effective amount is between about 10 to about 1×10¹⁶ total particles/kg of the agent.

15. The method according to claim 7, wherein the therapeutically effective amount is between about 10 to about 1×10¹⁶ VG/kg of the agent.


SEQUENCE LISTING (1 sequence)

SEQ ID NO: 1 - protein, 400 aa, Artificial Sequence
  Synthetic Sequence
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRKITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP